(19) United States
(12) Patent Application Publication
(10) Pub. No.: US 2020/0338088 A1
(43) Pub. Date: Oct. 29, 2020

(54) Methods for Treating Neutrophilic Dermatoses with SYK Inhibitors

(71) Applicant: Rush University Medical Center, Chicago, IL (US)

(72) Inventors: Andrei B. NESTEROVITCH, Orland Park, IL (US); Tibor T. GLANT, Chicago, IL (US); Michael D. THARP, Hinsdale, IL (US)

(73) Assignee: Rush University Medical Center, Chicago, IL (US)

(21) Appl. No.: 16/958,924

(22) PCT Filed: Jan. 4, 2019
(86) PCT No.: PCT/US2019/012280

§ 371 Date: Jun. 29, 2020

Related U.S. Application Data
(60) Provisional application No. 62/613,486, filed on Jan. 4, 2018.

(51) Int. Cl. A61K 31/5377; A61K 9/00; A61K 31/501; A61K 31/675; A61K 31/5383; A61K 31/505; A61K 31/519; A61K 31/437; A61K 31/05; A61K 31/4422

(52) U.S. Cl. CPC A61K 31/5377; A61K 9/0014; A61K 9/0053; A61K 31/501; A61K 31/675; A61K 31/4422; A61K 31/505; A61K 31/519; A61K 31/437; A61K 31/05; A61K 31/5383

(57) ABSTRACT

Methods of treating neutrophilic dermatoses and PTPN6 deficiencies in a subject are provided. The methods include administering a therapeutically effective amount of a pharmaceutical composition comprising spleen tyrosine kinase (SYK) inhibitor to the subject.

### RELATED APPLICATIONS

This application claims the benefit of U.S. Provisional Application No. 62/613,486, filed Jan. 4, 2018, which is incorporated by reference herein in its entirety.

### BACKGROUND

The present disclosure generally relates to pharmaceutical compositions and methods useful for the treatment of diseases and disorders. More particularly, the disclosure relates to pharmaceutical compositions and methods for treatment of neutrophilic dermatoses comprising SYK inhibitors.

Neutrophilic dermatoses (ND) are a rare, complex and heterogeneous group of skin diseases with overlapping clinical symptoms⁵⁻¹¹. Sterile infiltration of the skin by neutrophils is the most characteristic histopathological feature in Sweet's syndrome (SW, acute febrile ND), pyoderma gangrenosum (PG), PAPA (pyogenic arthritis, pyoderma gangrenosum and acne) syndrome, SAPHO (synovitis, acne, pustulosis, hyperostosis, osteitis) syndrome, rheumatoid neutrophilic dermatitis, sterile neutrophilic folliculitis, Majeed syndrome, neutrophilic eccrine hidradenitis PAPASH/PASH (pyrogenic arthritis, pyoderma gangrenosum, acne, and hidradentitis) syndromes and psoriasis⁶⁻¹¹. ND are characterized by (i) the presence of neutrophil infiltrates in the skin, (ii) the lack of infectious etiology, (iii) frequent extracutaneous manifestations of neutrophilic infiltration, (iv) association with other systemic (myeloproliferative, autoimmune, or autoinflammatory) disorders, and (v) high responsiveness to steroid or immunosuppressive treatment.

SW is a rare disease (prevalence <1 in 1,000,000 people) with different clinical forms¹¹. Although numerous case reports, clinical/research studies and reviews have been published (over 1,800 PubMed search hits), the etiology of SW is unknown. The disease may be (i) idiopathic in 70% of the cases, (ii) associated with malignancies (particularly myeloid leukemia) in 10-20% of cases, (iii) associated with autoimmune or autoinflammatory disorders such as IBD, Behcet's disease, rheumatoid arthritis, sarcoidosis, ankylosing spondylitis, etc., and (iv) drug-induced¹¹.

PG is also a rare disease. The exact prevalence of PG is unknown. The incidence has been estimated to range between 1 and 3.3 per 330,000 people¹². It is also frequently associated with inflammatory bowel diseases (˜30% of PG patients have or will develop IBD), rheumatoid arthritis, ankylosing spondylitis, myeloid leukemia, myeloma and lymphoma. The disease rapidly leads to skin ulceration, and (like SW), responds well to high-dose steroid or immunosuppressive treatment. Despite extensive literature on the subject (over 2,700 PubMed search results), the etiology of PG is also unknown, and the diagnosis most likely includes different subtypes of the disease^{13,14}. Familial cases of both SW and PG are not frequent and occur as inherited autoinflammatory diseases¹⁵⁻²⁰.

### BRIEF SUMMARY

Methods of treating neutrophilic dermatoses and Ptpn6 deficiencies in a subject are provided. The methods include administering a therapeutically effective amount of a pharmaceutical composition comprising spleen tyrosine kinase (SYK) inhibitor to the subject.

### FIGURE DESCRIPTION

FIG. 1 illustrates relative fluorescence levels (phosphatase PTPN6 activities) in leukocyte cell lysates of patients with pyoderma gangrenosum PG2 through PG9 compared with four normal samples. All PG samples expressed significantly reduced (*p<0.05) phosphatase activity (relative fluorescence units) compared with any of the four healthy individuals.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Methods of treating neutrophilic dermatoses in a subject are provided. In some embodiments, the methods include administering a therapeutically effective amount of a spleen tyrosine kinase (SYK) inhibitor to a subject.

“Treating”, “treat”, or “treatment” within the context of the instant invention, means an alleviation of symptoms associated with a disorder or disease, or halt of further progression or worsening of those symptoms, or prevention or prophylaxis of the disease or disorder. For example, within the context of this invention, successful treatment may include an alleviation of symptoms related to cancer and/or folic acid deficiency.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a neutrophilic dermatosis), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The term “subject” or “patient” as used herein, refers to a mammal, preferably a human.

Neutrophilic Dermatoses (ND)

Neutrophilic dermatoses (ND) are autoinflammatory skin conditions characterized by infiltration of inflammatory cells (neutrophils) in the affected tissue with no evidence of infection. Non-limiting examples of ND include Sweet's syndrome (SW, acute febrile ND), pyoderma gangrenosum (PG), PAPA (pyogenic arthritis, pyoderma gangrenosum and acne) syndrome, SAPHO (synovitis, acne, pustulosis, hyperostosis, osteitis) syndrome, rheumatoid neutrophilic dermatitis, sterile neutrophilic folliculitis, Majeed syndrome, neutrophilic eccrine hidradenitis PAPASH/PASH (pyrogenic arthritis, pyoderma gangrenosum, acne, and hidradentitis) syndromes and psoriasis.

Spleen Tyrosine Kinase

Spleen tyrosine kinase (SYK) is an intracellular cytoplasmic tyrosine kinase that is an important mediator of immunoreceptor signaling in macrophages, neutrophils, mast cells, and B cells. By “Syk biological activity” is meant any of the following activities: kinase activity, transmission of signals from cell receptors including B-cell, T-cell and Fc receptors, integrins, and CD74, or cell signaling including, for example, osteoclast, monocyte, or mast cell signaling.

Spleen Tyrosine Kinase Inhibitors

A Spleen Tyrosine Kinase Inhibitor “SYK inhibitor” refers to any agent that inhibits the biological activity of SYK or expression of SYK. A SYK inhibitor may inhibit the kinase activity of SYK. The SYK inhibitors may include small molecules, antibodies or antigen-binding fragments thereof, nucleic acids, nucleic acid analogs or derivatives, peptides, peptidomimetic, a protein, a monosaccharide, a disaccharide, a trisaccharide, an oligosaccharide, a polysaccharide, a lipid, a glycosaminoglycan, an extract made from a biological material, and combinations thereof.

By way of non-limiting example, the SYK inhibitor may be selected from 6-(1H-indazol-6-yl)-N-(4-morpholin-4-ylphenyl)imidazo[1,2-a]pyrazin-8-amine (Entospletinib, GS-9973, CAS 1229208-44-9), 6-[(1R,2S)-2-Amino-cyclohexylamino]-4-(5,6-dimethyl-pyridin-2-ylamino)-pyridazine-3-carboxylic acid amide (R09021), 6-({5-Fluoro-2-[(3,4,5-trimethoxyphenyl)amino]pyrimidin-4-yl}amino)-2,2-dimethyl-3-oxo-2,3-dihydro-4H-pyrido[3,2-b][1,4]oxazin-4-yl]methyl dihydrogen phosphate, (Fostamatinib, R788, CAS 901119-35-5), N4-(2,2-dimethyl-3-oxo-4H-pyrid[1,4]oxazin-6-yl)-5-fluoro-N2-(3,4,5-trimethoxyphenyl)-2,4-pyrimidinediamine (tamatinib, R406, CAS 841290-80-0), 3,3′-[(5-Fluoro-2,4-pyrimidinediyl)diimino]bis[phenol]; 3,3′-[(5-Fluoro-2,4-pyrimidinediyl)diimino]bis-phenol (R112-CAS 575474-82-7) 2-[[7-(3,4-dimethoxyphenyl)imidazo[1,2-c]pyrimidin-5-yl]amino]pyridine-3-carboxamide (BAY 61-3606, CAS 732983-37-8), 4-(cyclopropylamino)-2-[[4-[4-(ethylsulfonyl)-1-piperazinyl]phenyl]amino]-5-pyrimidinecarboxamide (cerdulatinib, CAS 1198300-79-6), TAK-659, (Takeda), and 4-[(E)-2-(3,5-dihydroxyphenyl)ethenyl]benzene-1,2-diol (piceatannol, CAS 10083-24-6) and 5-isopropyl-3-methyl-2-cyano-6-methyl-4-(3-nitrophenyl)-1,4-dihydro-3,5-pyridinecarboxylate (nilyadipine, CAS 75530-68-6). Other SYK inhibitors may also be used.

The SYK inhibitor may be administered alone or in combination with one or more additional therapeutic agents. Standard oral dosages of the SYK inhibitor used in clinical trails may be suitable for treating ND. By way of non-limiting example, an oral dose of 10 mg/kg of entospletinib has been used in Phase I safety trials and an oral dose of 1600 mg/day has been used in Phase II trials in chronic lymphocytic leukemia. In some embodiments, the SYK inhibitor may be administered topically. For example, small single molecule water soluble drugs with low molecular weight<500 kDa may be delivered by topical application, but topical application is not limited to these drugs. Entospletinib is an example of a small single molecule drug with low molecular weight (411 kDa) that may be delivered by topical application. R09021 is another example of a small single molecule drug with low molecular weight (355 kDa) that may be delivered by topical application.

In certain embodiments, a pharmaceutical composition may be provided. In a related embodiment, a pharmaceutical composition of any of the compositions of the present invention and a pharmaceutically acceptable carrier or excipient of any of these compositions may be provided.

In one embodiment, a packaged treatment may be provided. The packaged treatment includes a composition of the invention packaged with instructions for using an effective amount of the composition of the invention for an intended use. In other embodiments, a use of any of the compositions for manufacture of a medicament to treat a neutrophilic dermatosis in a subject is provided.

Pharmaceutical Compositions

The compositions described herein may be used alone or in compositions together with a pharmaceutically acceptable carrier or excipient. Pharmaceutical compositions of the present invention comprise a therapeutically effective amount of a Spleen Tyrosine Kinase (SYK) inhibitor and may further include one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds described herein may be administered to humans and animals in dosage unit formulations containing conventional nontoxic pharmaceutically acceptable carriers, adjuvants, and vehicles as desired.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules or lipid particles, lyophilized powders, or other forms known in the art.

Compositions of the invention may be formulated for delivery as a liquid aerosol or inhalable dry powder. Liquid aerosol formulations may be nebulized predominantly into particle sizes that can be delivered to the terminal and respiratory bronchioles.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

Aerosolized formulations of the invention may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 15 μm. Predominantly in this application means that at least 70% but preferably more than 90% of all generated aerosol particles are within 15 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

A compound described herein can be administered alone or in combination with other compounds, for a possible combination therapy being staggered or given independently of one another. Long-term therapy is equally possible as is adjuvant therapy in the con-text of other treatment strategies, as described above. Other possible treatments are therapy to maintain the patient's status after the initial treatment, or even preventive therapy, for example in patients at risk.

Effective amounts of the compounds of the invention generally include any amount sufficient to detectably an inhibition or alleviation of symptoms. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### EXAMPLES

A spontaneous mutation in the protein tyrosine phosphatase, non-receptor type-6 (Ptpn6) gene in mice has been identified. All animals with a homozygous mutation Ptpn6^{meb2/meb2} showed clinical and histopathological similarities to human neutrophilic dermatoses. The Ptpn6^{meB2/meB2} mice the Ptpn6 gene had the B2 repeat insertion that created a functionally impaired enzyme leading to a deficit in neutrophil apoptosis. The most prominent abnormalities in Ptpn6^{meB2/meB2} homozygous mice include massive leukocytosis, altered myelopoiesis, and neutrophil infiltration in the skin and other organs, all of which could be suppressed by high-dose corticosteroid treatment. (Nesterovitch, A B et al., Am J Pathol 2011: 178: 1701-14.)

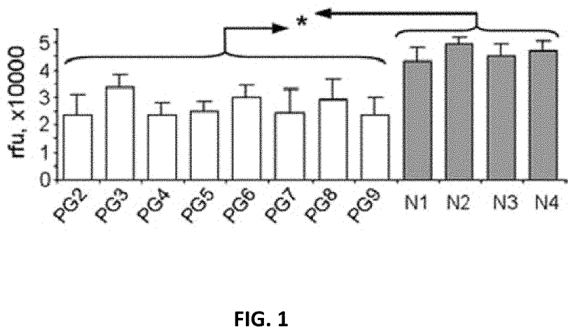

Other mutations in the Ptpn6 gene in mice have also been identified that cause development of Neutrophilic Dermatosis-Like Disease (NDLD). (me/mev mice: Shultz L D, Schweitzer P A, Rajan T V, Yi T, Ihle J N, Matthews R J, Thomas M L, Beier D R: Mutations at the murine motheaten locus are within the hematopoietic cell protein-tyrosine phosphatase (Hcph) gene. Cell 1993, 73:1445-1454; spin mice: Croker B A, Lawson B R, Rutschmann S, Berger M, Eidenschenk C, Blasius A L, Moresco E M, Sovath S, Cengia L, Shultz L D, Theofilopoulos A N, Pettersson S, Beutler B A: Inflammation and autoimmunity caused by a SHP1 mutation depend on IL-1, MyD88, and a microbial trigger. Proc Natl Acad Sci USA 2008, 105:15028-15033.) The mutations in the Ptpn6 gene cause decreased phosphatase activity (Ptpn6-insufficiency). To explore the potential involvement of PTPN6 in human diseases, blood samples were collected from consenting patients. (9 SW and 14 PG patients.) One patient with familial Sweet's syndrome carried a mutation (E441G) in exon 11 and a 1.7 Kbp deletion in the promoter region of the PTPN6 gene. (Nesterovitch, A B et al., Am J Pathol 2011: 178: 1434-41.) The patients with diagnosed idiopathic SW and PG expressed a number of functionally inactive spice variants, but very low or almost undetectable amounts of normal PTPN6 protein. PG samples had significantly (p<0.05) reduced phosphatase activity compared to health individuals (See FIG. 1). An independent study found that proteins in neutrophils of PG patients are hyperphosphorylated. No enzyme replacement therapy exists for PTPN6-insufficiency.

PTPN6 works as a negative regulator of signaling for multiple receptors of innate and adaptive immunities in both mice and humans. PTPN6 antagonizes SYK signaling. Ptpn6 dephosphorylates proteins which were phosphorylated by SYK kinase terminating signaling from multiple immunoreceptors. (Turner et al., Immunol Today, 21:148-54; Alsadeq et al., J Immunol 2014, 193: 268-76.) Mice with full length deletions of both the Ptpn6 and Syk genes in neutrophils (Ptpn6^{flfl}Syk^{fl/fl} S100a8-cre) are completely free of paw inflammation. (Abram et al., Immunity 2013, 38: 489-501.) This creates the opportunity for therapeutic rescue of Ptpn6-insufficiency with SYK inhibitors, for example small molecule SYK inhibitors that can be used for treatment of ND.

SYK inhibitors may also be used in diseases that have decreased expression of PTPN6. For example, PTPN6 expression is decreased in psoriasis (Eriksen, et al. J invest Dermatol 2010, 130: 1590-7) and psoriatic arthritis (Batliwalla, et al. Mol Med 2005, 11: 21-29). Psoriasis is a subtype of neutrophilic dermatoses with prominent neutrophilic infiltration of the epidermis. PTPN6 expression is also decreased in multiple sclerosis (Christophi et al, Lab Invest 2009, 89:742-59, Kumagai et al., J Neuroimmunol 2012, 246:51-7.), AML (Beghini et al, Hum Mol Genet 200, 9: 2297-304, Luo et al. Zhongguo Shi Yan Xue Ye Xue Za Zhi 2004; 12: 128-32.), diffuse large B-cell lymphoma (Demosthenous et al, Oncotarget 2015, 6: 44703-13.), and T-cell lymphoma (Zhang et al., Am J Pathol 2000, 157: 1137-46.).

Ptpn6^{meB2/meB2} mice will serve as an animal model of ND in humans. To date, four different spontaneous mutations have been identified in the mouse Ptpn6 gene in so called “motheaten” mice: me, mev, spin (in the C57BL6 genetic background) and meB2 (in the BALB/c genetic background). The clinical and histopathological abnormalities of the skin and other organs in Ptpn6^{meB2/meB2} mice are linked with those described in human ND patients. The severity and progression of the disease is different in all four Ptpn6-mutant mice. The most severe phenotype is observed in the classical “motheaten” mutation (Ptpn6^{me/me}), in which the maximum life span is 2-3 weeks. The Ptpn6^{meB2/meB2} mice have less severe and easily detectable (due to white fur of BALB/c genetic background) phenotype and a longer life span, which makes this model more suitable for laboratory experiments.

We will study the therapeutic potential of compensating Ptpn6-insufficiency using the selective SYK inhibitor (GS-9973, entospletinib) in Ptpn6^{meB2/meB2} mice. Homozygous Ptpn6^{meB2/meB2} mice will be treated with the SYK inhibitor, entospletinib (GS-9973), as a model for treatment for neutrophilic dermatosis (ND) in humans. Entospletinib (GS-9973) is the most selective ATP-competitive SYK inhibitor, which has already undergone Phase I trials on healthy volunteers⁴⁶, and Phase I I trials in chronic lymphocytic leukemia (CLL)³⁰. Treatments with inactivating antibodies were shown to be effective in a therapeutic inactivation of a particular receptor and its signaling. Our NDLD or motheaten mev mice were successfully treated with anti-Gr1, anti-CD44 and anti-CD11b antibodies^{1,47}. In this study, small molecule drugs that are less expensive to develop and thus are more affordable for patients than large molecule biologics. Moreover, a small single molecule drug with low molecular weight can potentially be delivered by topical application, which is preferable in a dermatologic practice.

Six 5-week-old Ptpn6^{meB2/meB2} mice with NDLD and six healthy Ptpn6^{wt/wt} littermate mice will be injected with daily subcutaneous dose of 10 μg/g of SYK inhibitor entospletinib (GS-9973) for 14 days (dissolved as recommended by the manufacturer for in vivo use in 4 μl DMSO, 30 μl PEG 300, 5 μl Tween 80 and 100 μl PBS)^{48,49}. The mice will be sacrificed and the serum levels of entospletinib (GS-9973), pSyk activity²⁹ and the paw inflammation score¹ at the end of the study (after 14 days) will be measured. Second, six 5-week-old Ptpn6^{meB2/meB2} with NDLD and six healthy Ptpn6^{wt/wt} littermate mice will be treated with a daily oral dose of 10 μg/g of SYK inhibitor entospletinib (GS-9973) for 14-30 days and the serum level of entospletinib (GS-9973), pSyk activity²⁹ and the paw inflammation score¹ at the end of the study will be measured. The results of these experiments will be compared to our previous treatments of NDLD mice with oral prednisone, gliotoxin, anti-CD44 and anti-Gr1 monoclonal antibodies¹.

The daily oral dose of 10 mg/kg (10 μg/g) of GS-9973 significantly inhibited ankle inflammation in a rat collagen-induced arthritis model after 7 days²⁹. An oral dose of 1600 mg/day (estimated 20 mg/kg per day) was used in a human Phase 2 trial for 24 weeks in CLL³⁰. If no improvement in the clinical phenotype and pSyk activity>40-50% is observed within 14 days, we will increase the dose to 20-40 μg/g daily. If there is an observed increase in mortality in Ptpn6^{meB2/meB2} and/or the healthy Ptpn6^{wt/wt} littermate mice, the dose will be decreased to 5 μg/g daily. The treatments with additional mice will be conducted to increase the statistical power of the study. We propose to have at least 16-18 animals in each study group.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A method of treating neutrophilic dermatoses (ND) in a subject comprising:
administering a therapeutically effective amount of a pharmaceutical compositions comprising spleen tyrosine kinase (SYK) inhibitor to the subject in need thereof.

2. The method according to claim 1, wherein the SYK inhibitor is a small molecule having a molecular weight of <500 kDa.

3. The method according to claim 1, wherein the pharmaceutical composition is administered topically.

4. The method according to claim 1, wherein the pharmaceutical composition is administered orally.

5. The method according to claim 1, wherein the pharmaceutical composition is formulated together with a pharmaceutically acceptable carrier or excipient.

6. The method according to claim 1, wherein the SYK inhibitor is selected from the group consisting of entospletinib, RO9021, Fostamatinib, tamatinib, R112, BAY 61-3606, TAK-659, piceatannol and nilyadipine.

7. The method according to claim 1, wherein the ND is selected from the group consisting of Sweet's syndrome (SW, acute febrile ND), pyoderma gangrenosum (PG), PAPA (pyogenic arthritis, pyoderma gangrenosum and acne) syndrome, SAPHO (synovitis, acne, pustulosis, hyperostosis, osteitis) syndrome, rheumatoid neutrophilic dermatitis, sterile neutrophilic folliculitis, Majeed syndrome, neutrophilic eccrine hidradenitis PAPASH/PASH (pyrogenic arthritis, pyoderma gangrenosum, acne, and hidradentitis) syndromes, psoriatic arthritis and psoriasis.

8. A method of treating a PTPN6 deficiency in a subject comprising:
administering a therapeutically effective amount of a pharmaceutical composition comprising spleen tyrosine kinase (SYK) inhibitor to the subject in need thereof.

9. The method according to claim 8, wherein the SYK inhibitor is a small molecule having a molecular weight of <500 kDa.

10. The method according to claim 8, wherein the SYK inhibitor is selected from the group consisting of entospletinib, R09021, fostamatinib, tamatinib, R112, BAY 61-3606, TAK-659, piceatannol and nilyadipine.

11. The method according to claim 8, wherein the subject has a pathologic process characterized by neutrophil involvement.

12. The method according to claim 11, wherein the pathologic process is selected from the group consisting of neutrophilic dermatosis, psoriatic arthritis, multiple sclerosis, acute myeloid leukemia (AML), diffuse large B-cell lymphoma and T-cell lymphoma.
